(19) United States
(12) Patent Application Publication
(10) Pub. No.: US 2012/0179236 A1
(43) Pub. Date: Jul. 12, 2012

(54) SIDEPORT ENGAGEMENT AND SEALING MECHANISM FOR ENDOLUMINAL STENT-GRAFTS

(75) Inventors: Rafael Benary, Tel Aviv (IL); Alon Shalev, Ra'anana (IL)

(73) Assignee: ENDOSPAN LTD., Netanya (IL)

(21) Appl. No.: 13/384,075

(22) PCT Filed: Jul. 14, 2010
(86) PCT No.: PCT/IL10/00564

§ 371 Date: Mar. 26, 2012

Related U.S. Application Data
(60) Provisional application No. 61/225,228, filed on Jul. 14, 2009.

(51) Int. Cl. A61F 2/82; B23P 11/00

(52) U.S. Cl. 623/1.13; 29/428

(57) ABSTRACT

An endovascular prosthesis (10) includes first and second endovascular stent-grafts (20, 22), which include respective first and second stent bodies (32, 108), and respective first and second fluid flow guides (102, 109). The first stent-graft (20) is shaped to define an interface portion (103) having a distal interface end (34) that meets a proximal end (36) of the first stent body (32) at a peripheral juncture (104). The second stent-graft (22) defines an interface aperture (110), within which part of the interface portion (103) is positionable. The interface portion (103) comprises engagement support members (105) disposed around a periphery thereof, which are configured to transition from an initial state to (b) a sealing state, thereby sealingly coupling the first stent-graft (20) to the second stent-graft (22) when the part of the interface portion (103) is positioned within the interface aperture (110). Other embodiments are also described.

### CROSS-REFERENCE TO RELATED APPLICATIONS

The present patent application claims priority from U.S. Provisional Application 61/225,228, filed Jul. 14, 2009, entitled, “Sideport engagement and sealing mechanism for endoluminal stent grafts,” which is incorporated herein by reference.

### FIELD OF THE APPLICATION

This present application relates generally to prostheses and surgical methods, and specifically to tubular prostheses, including endovascular grafts and stent-grafts.

### BACKGROUND OF THE APPLICATION

Endovascular prostheses are sometimes used to treat aortic aneurysms. Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”) and aortic uni-iliac (“AUI”) aneurysms.

PCT Publication WO 2008/107885 to Shalev et al., and US Patent Application Publication 2010/0063575 to Shalev et al. in the US national stage thereof, which are incorporated herein by reference, describe a multiple-component expandable endoluminal system for treating a lesion at a bifurcation, including a self expandable tubular root member having a side-looking engagement aperture, and a self expandable tubular trunk member comprising a substantially blood impervious polymeric liner secured therealong. Both have a radially-compressed state adapted for percutaneous intraluminal delivery and a radially-expanded state adapted for endoluminal support.

The following references may be of interest:

U.S. Pat. No. 4,938,740 to Melbin

U.S. Pat. No. 5,824,040 to Cox et al.

U.S. Pat. No. 7,044,962 to Elliott

US Patent Application Publication 2006/0229709 to Morris et al.

US Patent Application Publication 2006/0241740 to Vardi et al.

US Patent Application Publication 2008/0109066 to Quinn

### SUMMARY OF APPLICATIONS

In some applications of the present invention, an endovascular prosthesis comprises first and second endovascular stent-grafts. The first and second stent-grafts are configured to be sealingly coupled together, in order to define a fluid flow path through both stent-grafts. The first and second stent-grafts comprise respective structural members, which define respective stent bodies, which are generally tubular when the stent-grafts assume radially-expanded states. The first and second stent-grafts further comprise respective first fluid flow guides, which are coupled to the respective stent bodies, in order to define respective fluid flow paths therethrough.

The first stent-graft is shaped so as to define an interface portion, which has a distal interface end that meets a proximal end of the first stent body at a peripheral juncture. Typically, the first fluid flow guide covers at least a covered portion of the interface portion, which helps seal the first stent-graft to the second stent-graft. The first stent-graft further comprises a plurality of engagement support members disposed around a periphery of the interface portion. Optionally, the engagement support members are elongated, and may be shaped as arms. The engagement support members are typically configured to transition from an initial state to a sealing state. For some applications, the engagement support members are configured to extend proximally when in the initial state, and to extend distally toward a distal end of the first stent body when in the sealing state.

The structural member and fluid flow guide of the second stent-graft together define an interface aperture at a location other than at ends the second stent-graft, when the second stent-graft assumes a radially-expanded state. The interface portion of the first stent-graft and the interface aperture of the second stent-graft are configured such that part of the interface portion is positionable within the interface aperture. When the part of the interface portion is thus positioned within the interface aperture, and the engagement support members assume the sealing state, the engagement support members sealingly couple the first stent-graft to the second stent-graft, thereby preventing fluid from leaking between the two prostheses at the interface aperture.

Typically, the engagement support members and the second stent-graft are configured such that the engagement support members internally press against a surface of the second stent-graft surrounding the interface aperture. This pressing helps seal the first stent-graft to the second stent-graft, thereby creating a continuous fluid flow path through the first and second stent-grafts.

For some applications, a radially-outwardly sloped portion of the first stent body serves as a sealing countersurface covered by the first fluid flow guide near the peripheral juncture. Typically, at least a portion of the sealing countersurface contacts an external surface of the second stent-graft surrounding the interface aperture, when the part of the interface portion is positioned within the interface aperture. The engagement support members and the second stent-graft are typically configured such that the engagement support members and the sealing countersurface sandwich a surface of the second stent-graft surrounding the interface aperture.

For some applications, the second fluid flow guide is shaped so as to define a radially-outward bulge at least partially surrounding the interface aperture, when the second stent-graft assumes its radially-expanded state. When the interface portion is positioned within the interface aperture, the bulge extends distally toward the first stent-graft. For applications in which the first stent-graft provides the sealing countersurface, the bulge typically contacts the sealing countersurface when the first stent-graft is sealingly coupled to the second stent-graft.

For some applications, the first stent-graft further comprises a non-covered portion that extends proximally beyond the interface portion, and which is pervious to fluids. The non-covered portion helps hold the first stent-graft in place within the second stent-graft.

For some applications, the fluid flow guide of the first stent-graft at least partially covers the engagement support members. The covered portions help seal the first stent-graft to the second stent-graft, in order to create a continuous, substantially fluid-impervious fluid flow path through the first and second stent-grafts. For some applications, the first stent-graft extends between at least two of the engagement support members that are circumferentially adjacent each other, when the first stent-graft assumes its radially-expanded state. These extension portions (which are similar to webbing) help seal the first stent-graft to the second stent-graft.

For some applications, the prosthesis is deployed in the descending aorta and both iliac arteries at the aorto-iliac junction. The second stent-graft is first deployed in both left iliac arteries, such that the interface aperture is aligned with the aorto-iliac bifurcation. In order to implant the first stent-graft, the first stent-graft is transvascularly (typically percutaneously) introduced into the aorta via one of the iliac arteries, while the first stent-graft is in its radially-compressed state positioned in a delivery catheter. The delivery catheter is advanced over a guidewire, and through one of the sides of the second stent-graft and the interface aperture, until the delivery catheter is positioned in the descending aorta. The delivery catheter is withdrawn proximally, allowing the first stent-graft to assume its radially-expanded state. The first stent-graft is manipulated until part of the interface portion is positioned within the interface aperture, and both the first and second stent-graphs are in their fully-deployed states. The engagement support members assume the sealing state, such that the engagement support members sealingly couple the first stent-graft to the second stent-graft, thereby preventing fluid from leaking between the two prostheses at the interface aperture.

More generally, for some applications, the prosthesis is deployed at a bifurcation between (a) the one or two first blood vessels and (b) a second blood vessel. The second stent-graft, while in its radially-compressed state, is transvascularly introduced into the one or two first blood vessels, such that the second stent-graft spans the bifurcation. The second stent-graft is transitioned to its radially-expanded state, such that the interface aperture is positioned at the bifurcation. The first stent-graft, while in its radially-compressed state, is transvascularly introduced into the second blood vessel via the interface aperture. The interface portion of the first stent-graft is positioned within the interface aperture. The first stent-graft is transitioned to its radially-expanded state, such that the engagement support members transition from the initial state to the sealing state, thereby sealingly coupling the first stent-graft to the second stent-graft.

For some applications, the first and second fluid flow guides together define a continuous fluid flow path that begins at a distal end of the first fluid flow guide, passes through the interface aperture, bifurcates proximally to the interface aperture, and passes through both ends of the second fluid flow guide. The fluid flow path is continuous because the first fluid flow guide is sealingly coupled to the second fluid flow guide. For some applications, the endovascular prosthesis is configured such that the fluid flow path provides substantially equal fluid flow through both ends of the second fluid flow guide.

There is therefore provided, in accordance with an application of the present invention, apparatus including an endovascular prosthesis, which includes:

first and second endovascular stent-grafts, which are configured to transition from respective radially-compressed states to respective radially-expanded states, and which include:
first and second structural members, respectively, at least respective portions of which define first and second stent bodies, which are generally tubular when the first and second stent-grafts assume the respective radially-expanded states; and
first and second fluid flow guides, respectively, which are coupled to the first and second stent bodies, respectively, so as to cover at least respective portions of the first and second stent bodies,

wherein the first structural member has proximal and distal ends, and is shaped so as to define an interface portion having a distal interface end that meets a proximal end of the first stent body at a peripheral juncture,

wherein, when the second stent-graft assumes its radially-expanded state, the second stent-graft is shaped so as to define an interface aperture at a location other than at ends of the second stent-graft,

wherein the interface portion and the interface aperture are configured such that part of the interface portion is positionable within the interface aperture, and

wherein the interface portion includes a plurality of engagement support members disposed around a periphery of the interface portion, which engagement support members are configured to transition from an initial state to a sealing state, thereby sealingly coupling the first stent-graft to the second stent-graft when the part of the interface portion is positioned within the interface aperture.

For some applications, the engagement support members meet the interface portion axially within a distance of the peripheral juncture, which distance equals 0.5 times an average diameter of the first stent body.

For some applications, the engagement support members are configured to assume the initial state when radially compressed, and the sealing state when radially relaxed.

For some applications, the apparatus further includes a delivery catheter, in which the first stent-graft is initially positioned, which delivery catheter is configured to hold the engagement support members in the initial state.

For some applications, a portion of at least some of the engagement support members is distally convex.

For some applications, the engagement support members and the second stent-graft are configured such that the engagement support members internally press against a surface of the second stent-graft surrounding the interface aperture, when the part of the interface portion is positioned within the interface aperture, the first and second stent-grafts assume their respective radially-expanded states, and the engagement support members assume the sealing state.

For some applications, the second fluid flow guide is shaped so as to define a radially-outward bulge at least partially surrounding the interface aperture, when the second stent-graft assumes its radially-expanded state.

For some applications, the first and second fluid flow guides include first and second biologically-compatible substantially fluid-impervious flexible sheets, respectively.

For some applications, the first structural member includes a self-expanding material. Alternatively or additionally, the first structural member may include a super-elastic alloy, such as Nitinol. For some applications, the second structural member includes a self-expanding material. Alternatively or additionally, the second first structural member may include a super-elastic alloy, such as Nitinol.

For some applications, the prosthesis further includes one or more radiopaque markers, disposed on the first structural member, and/or disposed on respective ones of the engagement support members.

For some applications, the location of a geometric center of the interface aperture is axially within a distance of an axial midpoint between the ends of the second stent-graft, which distance is 0.5 times an average diameter of the second stent body.

For some applications, the first and second structural members include first structural stent elements and second structural stent elements, respectively.

For any of the applications described above, the first fluid flow guide may cover at least a covered portion of the interface portion. For some applications, the covered portion of the interface portion extends proximally beyond the peripheral juncture by between 0.1 and 0.5 times an average diameter of the first stent body. Alternatively, the covered portion of the interface portion may extend proximally beyond the peripheral juncture by no more than 0.3 times an average diameter of the first stent body.

For any of the applications described above, the engagement support members may be configured to extend proximally when in the initial state, and to extend distally toward a distal end of the first stent body when in the sealing state.

For any of the applications described above, a perpendicular cross-sectional area of a portion of the first stent body covered by the first fluid flow guide may increase from the peripheral juncture in a direction toward a distal end of the first stent body, such that the portion of the first stent body serves as a sealing countersurface, which contacts an external surface of the second stent-graft surrounding the interface aperture when the part of the interface portion is positioned within the interface aperture, the first and second stent-grafts assume their respective radially-expanded states, and the engagement support members assume the sealing state. For some applications, the engagement support members and the second stent-graft are configured such that the engagement support members and the sealing countersurface sandwich a surface of the second stent-graft surrounding the interface aperture, when the part of the interface portion is positioned within the interface aperture, the first and second stent-grafts assume their respective radially-expanded states, and the engagement support members assume the sealing state. For some applications, the perpendicular cross-sectional area of the portion of the first stent body at an axial distance from the peripheral juncture is at least 30% greater than a perpendicular cross-sectional area of the peripheral juncture, which axial distance equals 0.3 times an average diameter of the first stent body, if the first and second stent-grafts were to assume their respective radially-expanded states with the interface portion not positioned within the interface aperture, and no force were exerted on the interface portion by either the first or the second stent-graft.

For any of the applications described above, a perpendicular cross-sectional area of the interface aperture may be between 65% and 85% of a greatest outer perpendicular cross-sectional area of the interface portion, if the first and second stent-grafts were to assume their respective radially-expanded states with the interface portion not positioned within the interface aperture, and no force were exerted on the interface portion by either the first or the second stent-graft. For some applications, the perpendicular cross-sectional area of the interface aperture is between 65% and 80% of the greatest outer perpendicular cross-sectional area of the interface portion, if the first and second stent-grafts were to assume their respective radially-expanded states with the interface portion not positioned within the interface aperture.

For any of the applications described above, the first stent-graft may include a non-covered portion that extends proximally beyond the interface portion, which non-covered portion includes a portion of the first structural member. For some applications, the non-covered portion has an axial length of at least 10 mm.

For any of the applications described above, the first fluid flow guide may at least partially cover the engagement support members. For some applications, when the first stent-graft assumes its radially-expanded state, the engagement support members are shaped so as to define respective radially-inward portions and radially-outward portions, and the first fluid flow guide covers the radially-inward portions but not the radially outward portions. For some applications, the first stent-graft extends between at least two of the engagement support members that are circumferentially adjacent each other, when the first stent-graft assumes its radially-expanded state.

For any of the applications described above, the engagement support members may be proximal engagement support members, the first stent-graft may further include a plurality of distal engagement support members, which are disposed more distally on the first stent-graft than are the proximal engagement support members, and the distal and proximal engagement support members may be configured to sandwich a surface of the second stent-graft surrounding the interface aperture, when the part of the interface portion is positioned within the interface aperture, the first and second stent-grafts assume their respective radially-expanded states, and the proximal engagement support members assume the sealing state.

For any of the applications described above, the interface aperture may be one of a plurality of interface apertures, the second stent-graft may be shaped so as to define the plurality of interface apertures at a respective plurality of locations other than at the ends of the second stent-graft, the first stent-graft may be one of a plurality of first stent-grafts, and the prosthesis may include a number of the first stent-grafts corresponding to a number of the interface apertures.

For any of the applications described above, the peripheral juncture may be generally elliptical, such as generally circular, when the first stent-graft assumes its radially-expanded state. For any of the applications described above, a perpendicular cross section of the interface aperture may be generally elliptical, such as generally circular, when the second stent-graft assumes its radially-expanded state.

For any of the applications described above, the first fluid flow guide and the second fluid flow guide may together define a continuous fluid flow path that begins at a distal end of the first fluid flow guide, passes through the interface aperture, bifurcates proximally to the interface aperture, and passes through both ends of the second fluid flow guide. For some applications, the endovascular prosthesis is configured such that the fluid flow path provides substantially equal fluid flow through both ends of the second fluid flow guide.

There is further provided, in accordance with an application of the present invention, apparatus including an endovascular stent-graft, which is configured to transition from a radially-compressed state to a radially-expanded state, and which includes:

a structural member, at least a portion of which defines a stent body, which is generally tubular when the stent-graft assumes the radially-expanded state; and

a fluid flow guide, which is coupled to the stent body, so as to cover at least a portion of the stent body,

wherein the structural member has proximal and distal ends, and is shaped so as to define an interface portion having a distal interface end that meets a proximal end of the stent body at a peripheral juncture,

wherein the interface portion includes a plurality of engagement support members disposed around a periphery of the interface portion, which engagement support members are configured to transition from an initial state to a sealing state.

For some applications, the fluid flow guide at least partially covers the engagement support members.

For some applications, when the stent-graft assumes the radially-expanded state, the engagement support members are shaped so as to define respective radially-inward portions and radially-outward portions, and the fluid flow guide covers the radially-inward portions but not the radially outward portions.

For some applications, the stent-graft extends between at least two of the engagement support members that are radially adjacent each other, when the stent-graft assumes the radially-expanded state.

For any of the applications described above, the engagement support members may be configured to extend proximally when in the initial state, and to extend distally toward a distal end of the first stent body when in the sealing state.

For any of the applications described above, the fluid flow guide may cover at least a covered portion of the interface portion. For some applications, the covered portion of the interface portion extends proximally beyond the peripheral juncture by between 0.1 and 0.5 times an average diameter of first stent body. Alternatively, the covered portion of the interface portion may extend proximally beyond the peripheral juncture by no more than 0.3 times an average diameter of the stent body.

For any of the applications described above, the apparatus may further includes a delivery catheter, in which the stent-graft is initially positioned, which delivery catheter is configured to hold the engagement support members in the initial state.

For any of the applications described above, the fluid flow guide may include at least one biologically-compatible substantially fluid-impervious flexible sheet.

For any of the applications described above, a perpendicular cross-sectional area of a portion of the stent body covered by the fluid flow guide may increase from the peripheral juncture in a direction toward a distal end of the stent body, such that the portion of the stent body serves as a sealing countersurface, when the stent-graft assumes the radially-expanded state.

For any of the applications described above, the engagement support members may be proximal engagement support members, and the stent-graft may further include a plurality of distal engagement support members, which are disposed more distally on the stent-graft than are the proximal engagement support members.

There is still further provided, in accordance with an application of the present invention, a method including:

providing first and second endovascular stent-grafts, which are configured to transition from respective radially-compressed states to respective radially-expanded states, and which include (a) first and second stent bodies, respectively, which are generally tubular when the first and second stent-grafts assume the respective radially-expanded states, and (b) first and second fluid flow guides, respectively, which are coupled to the first and second stent bodies, respectively, so as to cover at least respective portions of the first and second stent bodies, wherein the first structural member has proximal and distal ends;

transvascularly introducing the second stent-graft, while in its radially-compressed state, into one or two first blood vessels of a human subject, such that the second stent-graft spans a bifurcation between (a) the one or two first blood vessels and (b) a second blood vessel;

transitioning the second stent-graft to its radially-expanded state, such that an interface aperture defined by the second fluid flow guide is positioned at the bifurcation;

transvascularly introducing the first stent-graft, while in its radially-compressed state, into the second blood vessel via the interface aperture;

positioning an interface portion of the first stent-graft within the interface aperture, which interface portion has a distal interface end that meets a proximal end of the first stent body at a peripheral juncture; and

transitioning the first stent-graft to its radially-expanded state, such that a plurality of engagement support members thereof, which are disposed around a periphery of the interface portion, transition from an initial state to a sealing state, thereby sealingly coupling the first stent-graft to the second stent-graft.

For some applications, transitioning the first stent-graft includes transitioning the first stent-graft to its radially-expanded state, such that the plurality of engagement support members transition from (a) the initial state, in which the engagement support members extend proximally, to (b) the sealing state, in which the engagement support members extend distally toward a distal end of the first stent body.

For some applications, providing the first and second stent-grafts includes providing the first and second stent-grafts with respective first and second structural members, which include respective first structural stent elements and second structural stent elements, at least respective portions of which define the first and second stent bodies.

For some applications, the one or two first blood vessels are exactly one first blood vessel, and transvascularly introducing the second stent-graft includes transvascularly introducing the second stent-graft into the exactly one first blood vessel, such that the second stent-graft spans the bifurcation between the exactly one first blood vessel and the second blood vessel.

For some applications, the one or two first blood vessels are right and left iliac arteries, the second blood vessel is a descending aorta, and transvascularly introducing the second stent-graft includes transvascularly introducing the second stent-graft into the right and left iliac arteries, such that the second stent-graft spans the aorto-iliac bifurcation.

For some applications, transvascularly introducing the first stent-graft includes transvascularly introducing a delivery catheter, in which the first stent-graft is initially positioned, which delivery catheter is configured to hold the engagement support members in the initial state.

For some applications, transitioning the first stent-graft includes transitioning the first stent-graft to its radially-expanded state such that the engagement support members internally press against a surface of the second stent-graft surrounding the interface aperture.

For some applications, providing the first stent-graft includes providing the first stent-graft in which the first fluid flow guide covers at least a portion of the interface portion.

For some applications, providing the second fluid flow guide includes providing the second fluid flow guide shaped so as to define a radially-outward bulge at least partially surrounding the interface aperture, when the second stent-graft assumes its radially-expanded state.

For some applications, transitioning the second stent-graft includes transitioning the second stent-graft to its radially-expanded state such that a location of a geometric center of the interface aperture is axially within a distance of an axial midpoint between ends of the second stent-graft, which distance is 0.5 times an average diameter of the second stent body.

For some applications, providing the first stent-graft includes providing the first stent-graft in which a perpendicular cross-sectional area of a portion of the first stent body covered by the first fluid flow guide increases from the peripheral juncture in a direction toward a distal end of the first stent body, such that the portion of the first stent body serves as a sealing countersurface, and positioning the interface portion and transitioning the first stent-graft to its radially-expanded state includes causing an external surface of the second stent-graft surrounding the interface aperture to contact the sealing countersurface. For some applications, causing includes causing the engagement support members and the sealing countersurface to sandwich a surface of the second stent-graft surrounding the interface aperture.

For some applications, the first stent-graft includes a non-covered portion that extends proximally beyond the interface portion, which non-covered portion includes a portion of the first structural member, and transvascularly introducing the second stent-graft includes positioning the non-covered portion in exactly one of the one or two first blood vessels.

For some applications, providing the first stent-graft includes providing the first stent-graft in which the first fluid flow guide at least partially covers the engagement support members.

For some applications, providing the first and second stent-grafts includes providing the first and second stent-grafts in which the first fluid flow guide and the second fluid flow guide together define a continuous fluid flow path that begins at a distal end of the first fluid flow guide, passes through the interface aperture, bifurcates proximally to the interface aperture, and passes through both ends of the second fluid flow guide.

For some applications, the interface aperture is one of a plurality of interface apertures, the second stent-graft is shaped so as to define the plurality of interface apertures, the first stent-graft is one of a plurality of first stent-grafts, the second blood vessel is one of a plurality of second blood vessels, transvascularly introducing the first stent-graft includes transvascularly introducing a number of the first stent-grafts corresponding to a number of the interface apertures, into the second blood vessels, respectively, and positioning the interface portion includes positioning respective interface portions of the first stent-grafts within respective ones of the interface apertures.

The present invention will be more fully understood from the following detailed description of embodiments thereof, taken together with the drawings, in which:

### BRIEF DESCRIPTION OF THE DRAWINGS

FIG. 1 is a schematic illustration of an endovascular prosthesis, in accordance with an application of the present invention;

FIG. 2 is a schematic illustration of a first stent-graft of the prosthesis of FIG. 1 sealingly engaged with a second stent-graft of the prosthesis of FIG. 1, in accordance with an application of the present invention;

FIG. 3 is a schematic illustration of the deployment of two of engagement support members of the first stent-graft of FIG. 2, in accordance with an application of the present invention;

FIGS. 4A-D are schematic illustration of additional configurations of the first stent-graft of FIG. 1, in accordance with respective applications of the present invention;

FIG. 5 is a schematic illustration of the deployment of engagement support members of the configuration of the first stent-graft of FIGS. 4A-C, in accordance with an application of the present invention;

FIGS. 6A-D are schematic illustrations of an exemplary method of deploying the endovascular prosthesis of FIG. 1-3 or 4A-5, using an endovascular stent-graft delivery tool, in accordance with an application of the present invention;

FIGS. 6E-F schematically illustrate a method for extending the prosthesis of FIG. 1-3 or 4A-5, in accordance with an application of the present invention; and

FIG. 7 shows an exemplary fluid flow path through the endovascular prosthesis of FIG. 1-3 or 4A-5, in accordance with an application of the present invention.

### DETAILED DESCRIPTION OF APPLICATIONS

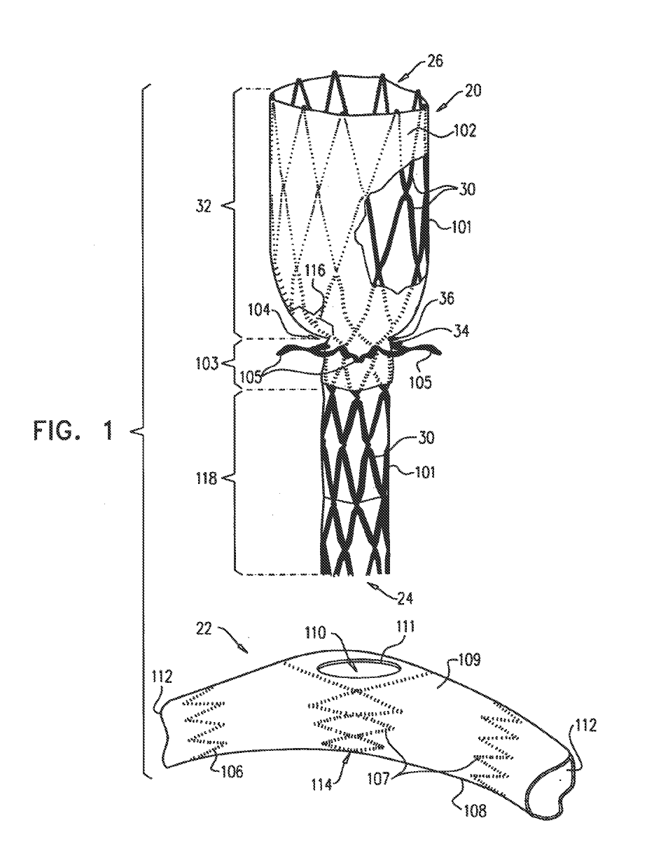

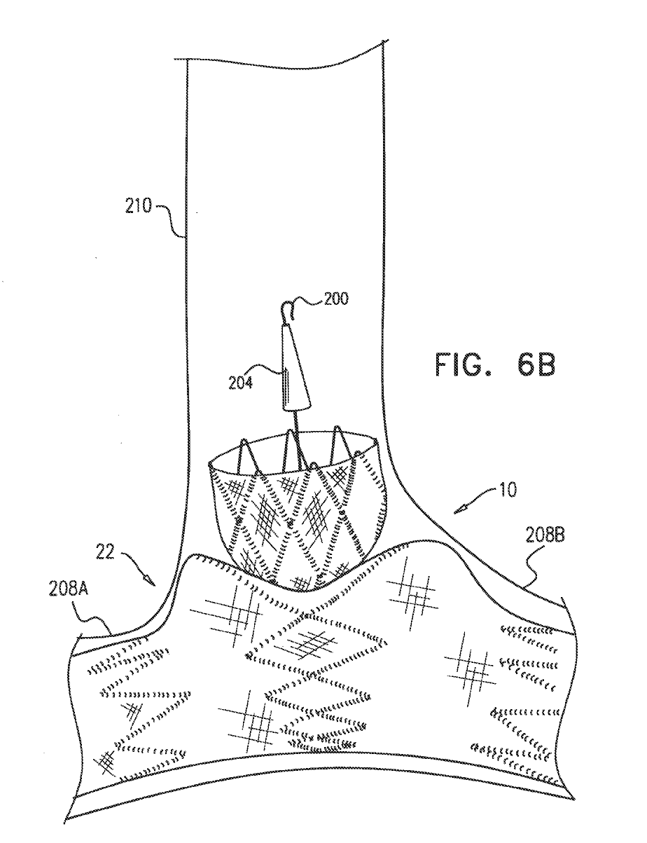

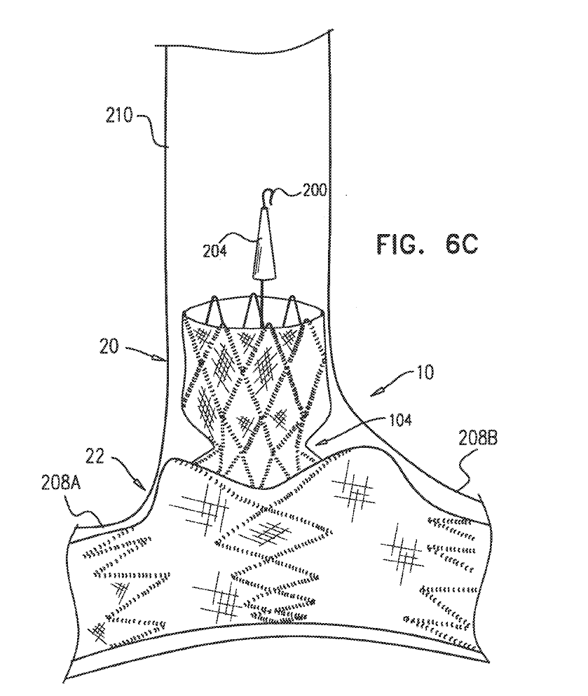

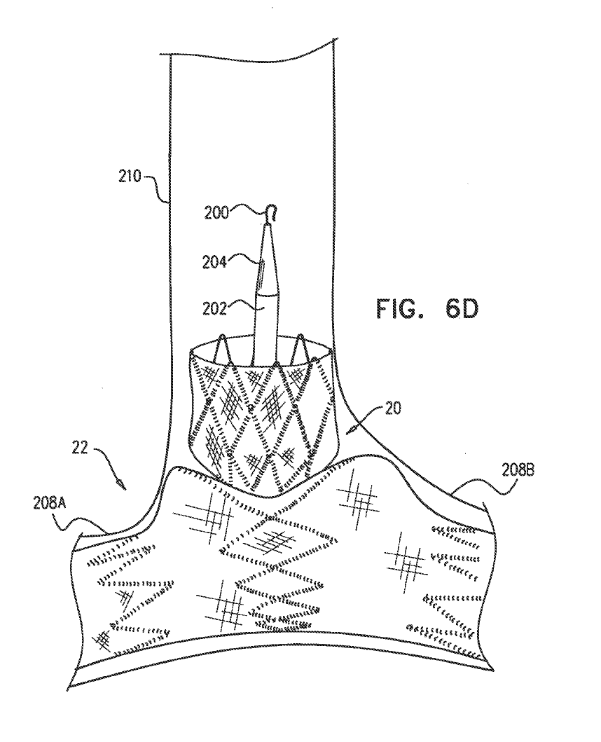

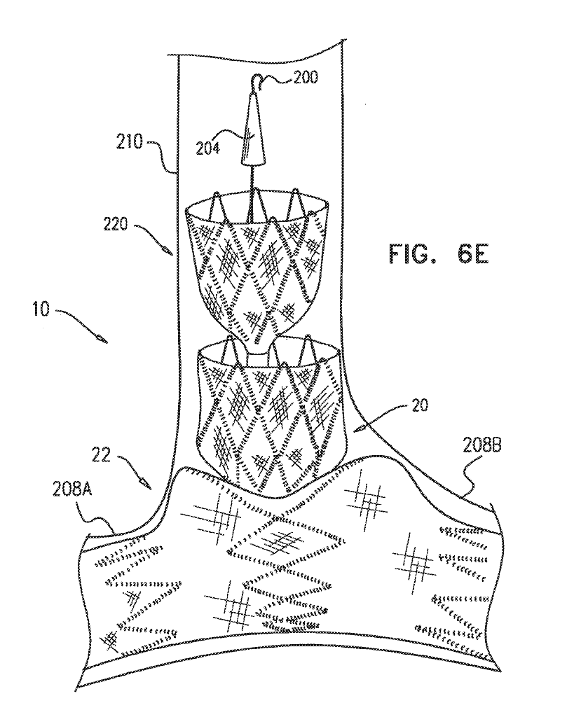

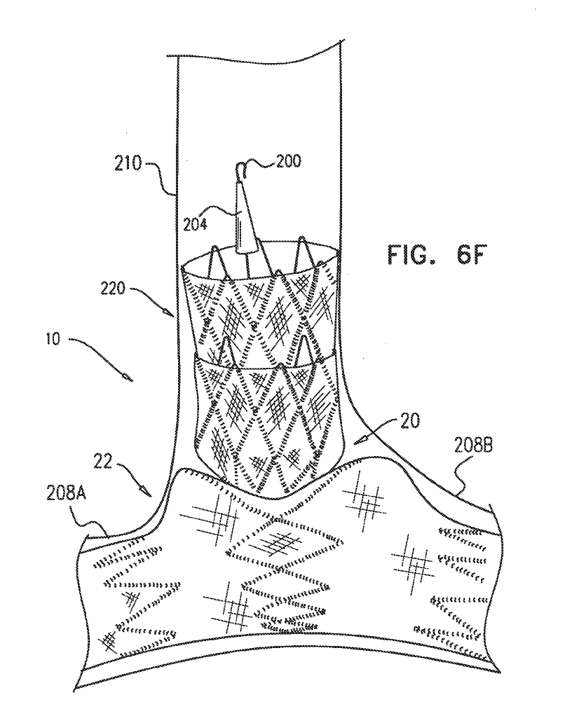

FIG. 1 is a schematic illustration of an endovascular prosthesis 10, in accordance with an application of the present invention. Prosthesis 10 comprises first and second endovascular stent-grafts 20 and 22, which are configured to transition from respective radially-compressed states, as described hereinbelow with reference to FIG. 6A, to respective radially-expanded states, as shown in FIGS. 1-3 and the other figures.

First stent-graft 20 has proximal and distal ends 24 and 26, and comprises a first structural member 101, which typically comprises first structural stent elements 30, at least a portion of which defines a first stent body 32, which is generally tubular when the first stent-graft assumes its radially-expanded state. First stent-graft 20 further comprises a first fluid flow guide 102, which is coupled to first stent body 32, such as by stitching, so as to cover at least a portion of the first stent body (i.e., to cover either an external or an internal surface of the at least a portion), in order to define a fluid flow path through the at least a portion. Typically, first fluid flow guide 102 comprises at least one biologically-compatible substantially fluid-impervious flexible sheet, which typically a fabric or textile. The flexible sheet may comprise, for example, a polymeric material (e.g., polytetrafluoroethylene), a textile material (e.g., polyethylene terephthalate (PET)), natural tissue (e.g., saphenous vein or collagen), or a combination thereof

First structural member 101 is shaped so as to define an interface portion 103, which has a distal interface end 34 that meets a proximal end 36 of the first stent body at a peripheral juncture 104. Typically, interface portion 103 is generally cylindrical when the first stent-graft assumes its radially-expanded state. Typically, peripheral juncture 104 is generally elliptical, such as generally circular, when the first stent-graft assumes its radially-expanded state.

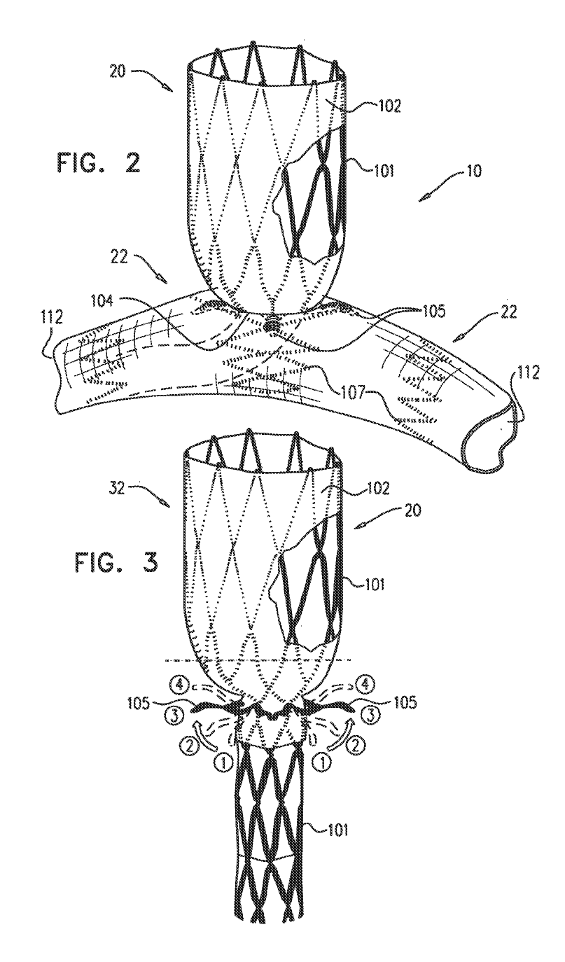

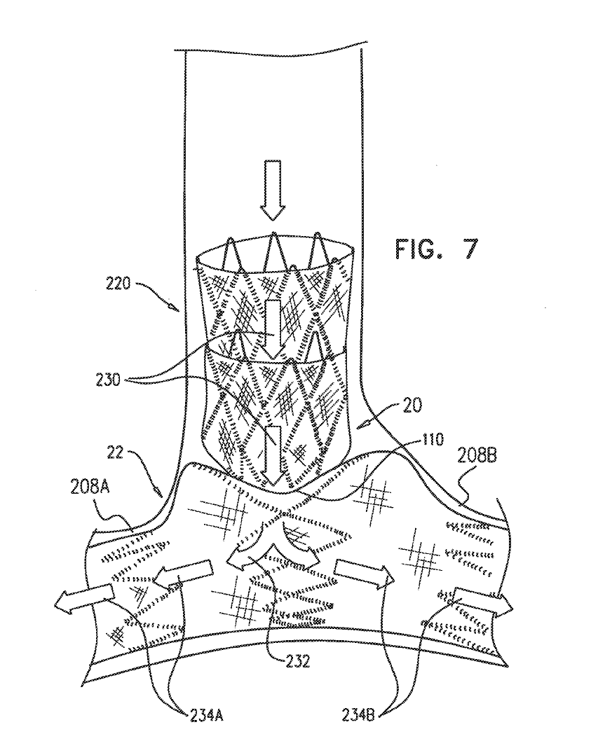

For some applications, first fluid flow guide 102 covers at least a covered portion of interface portion 103 (i.e., covers either an external or an internal surface of the covered portion). The covered portion helps seal first stent-graft 20 to second stent-graft 22, as described hereinbelow with reference to FIG. 2, in order to create a continuous, substantially fluid-impervious fluid flow path through the first and second stent-grafts, such as described hereinbelow with reference to FIG. 7. For some applications, as shown in FIG. 1, the covered portion of interface portion 103 extends proximally beyond peripheral juncture 104 by between 0.1 and 0.5 times an average diameter of first stent body 32.

For some applications, first stent-graft 20 further comprises a non-covered portion 118 that extends proximally beyond interface portion 103, and which is pervious to fluids. Non-covered portion 118 typically comprises a portion of first structural stent elements 30. Non-covered portion 118 helps hold first stent-graft 20 in place within second stent-graft 22. For example, the non-covered portion may have an axial length of at least 10 mm, no more than 300 mm, and/or between 10 and 300 mm, such as between 50 and 150 mm.

First stent-graft 20 further comprises a plurality of engagement support members 105 disposed around a periphery of interface portion 103. Optionally, the engagement support members are elongated, and may be shaped as arms. The engagement support members may comprise a portion of first structural stent elements 30. Typically, engagement support members 105 meet interface portion 103 axially within a distance of peripheral juncture 104, which distance equals 0.5 times an average diameter of first stent body 32, such as proximally adjacent the peripheral juncture. As described in more detail hereinbelow with reference to FIG. 3, engagement support members 105 are configured to transition from an initial state to a sealing state. Typically, the engagement support members extend proximally when in the initial state, and extend distally toward a distal end of first stent body 32 when in the sealing state (for example, the engagement support members may be inclined only slightly distally toward the distal end when in the sealing state). During the transition from the initial state to the sealing state, the engagement support members pass through intermediary states, as described hereinbelow with reference to FIG. 3. For clarity of illustration, the engagement support members are shown in FIG. 1 in one of these intermediary states (indicated by numeral 3 in FIG. 3).

Optionally, a portion (such a most radially-outward portion) of at least some of the engagement support members is distally convex (i.e., convex when viewed from a distal direction), such as to provide a large surface area pressing against second fluid flow guide 109 surrounding aperture 110. This inclination and shape generally reduce the wear and tear that the engagement support members may cause on second fluid flow guide 109. Optionally, the engagement support members additionally extend somewhat radially outward in the initial state, one or more of the intermediary states, and/or sealing state. Alternatively, for some applications, when in the sealing state, the engagement support members do not extend distally, but instead only extend more distally than when in the initial state, and/or extend more radially-outward than when in the initial state (which may be when the first stent-graft is in its radially-compressed state), without necessarily extending more distally than when in the initial state (which may be when the first stent-graft is in its radially-compressed state).

For some applications, engagement support members 105 are configured to assume the initial state when radially compressed, and the sealing state when radially relaxed. Typically, the engagement support members are initially compressed to assume the initial state by being positioned within a delivery catheter, such as described hereinbelow with reference to FIG. 6A.

Second stent-graft 22 comprises a second structural member 106, which typically comprises second structural stent elements 107, at least a portion of which defines a second stent body 108, which is generally tubular when the second stent-graft assumes its radially-expanded state. Second stent-graft 22 further comprises a second fluid flow guide 109, which is coupled to second stent body 108, such as by stitching, so as to cover at least a portion of the second stent body (i.e., to cover either an external or an internal surface of the at least a portion), in order to define two fluid flow paths through the at least a portion, such as described hereinbelow with reference to FIG. 7. Typically, second fluid flow guide 109 comprises at least one biologically-compatible substantially fluid-impervious flexible sheet, which typically a fabric or textile. The flexible sheet may comprise, for example, a polymeric material (e.g., polytetrafluoroethylene), a textile material (e.g., polyethylene terephthalate (PET)), natural tissue (e.g., saphenous vein or collagen), or a combination thereof.

When second stent-graft 22 assumes its radially-expanded state, the second stent-graft (typically, second structural member 106 and second fluid flow guide 109 together) defines an interface aperture 110 at a location other than at ends 112 of the second stent-graft. Interface aperture 110 serves as a sideport for sealingly coupling the second stent-graft with the first stent-graft. For some applications, as shown in the figures, the location of a geometric center of interface aperture 110 is axially within a distance of an axial midpoint 114 between ends 112 of second stent-graft 22, which distance is 0.5 times an average diameter of second stent body 108. For some applications, as shown in the figures, a perpendicular cross section of interface aperture 110 is generally elliptical, such as generally circular, when second stent-graft 22 assumes its radially-expanded state. In the present application, including in the claims, a “perpendicular cross section” is a planar cross section perpendicular to a longitudinal axis of the stent-graft.

For some applications, first structural member 101 comprises a self-expanding material, and/or a super-elastic alloy, such as Nitinol. For some applications, second structural member 106 comprises a self-expanding material, and/or a super-elastic alloy, such as Nitinol.

Reference is made to FIG. 2, which is a schematic illustration of first stent-graft 20 sealingly engaged with second stent-graft 22, in accordance with an application of the present invention. Interface portion 103 and interface aperture 110 are configured such that part of interface portion 103 is positionable within interface aperture 110. When the part of the interface portion is thus positioned within the interface aperture, and engagement support members 105 assume the sealing state, the engagement support members sealingly couple first stent-graft 20 to second stent-graft 22, thereby preventing fluid from leaking between the two prostheses at interface aperture 110. In FIG. 2, peripheral juncture 104 is the part of the interface portion that is positioned within interface aperture 110.

Typically, engagement support members 105 and second stent-graft 22 are configured such that the engagement support members internally press against a surface of the second stent-graft surrounding interface aperture 110, when the part of interface portion 103 is positioned within interface aperture 110, the first and second stent-grafts assume their respective radially-expanded states, and the engagement support members assume the sealing state. This pressing helps seal the first stent-graft to the second stent-graft, thereby creating a continuous fluid flow path through the first and second stent-grafts, such as described hereinbelow with reference to FIG. 7.

For applications in which first stent-graft 20 provides sealing countersurface 116, as described hereinbelow with reference to FIGS. 1 and 2, the engagement support members and the second stent-graft are typically configured such that the engagement support members and the sealing countersurface sandwich a surface of the second stent-graft surrounding the interface aperture (including a periphery 111 of interface aperture 110), when the part of the interface portion is positioned within the interface aperture, the first and second stent-grafts assume their respective radially-expanded states, and the engagement support members assume the sealing state.

For some applications, second stent-graft 22 is shaped so as to define a plurality of interface apertures 110, such as exactly two, exactly three, or four or more apertures.

Typically, a corresponding number of first stent-grafts 20 are provided, and are coupled to respective ones of the apertures. This multi-aperture configuration may be useful for implantation in a region of a blood vessel that has a plurality of bifurcations, such as the two bifurcations between the renal arteries and the descending aorta, or the three bifurcations between the aortic arch and the brachiocephalic trunk, common carotid artery, and subclavian artery, such as described hereinbelow.

For some applications, when the first and second stent-grafts assume their respective radially-expanded states, an average diameter of first stent-graft 20 is greater than an average diameter of second stent-graft 22. For example, these relative diameters may be appropriate for implantation at the aorto-iliac bifurcation. For other applications, the average diameter of the first stent-graft is less than the average diameter of the second stent-graft. For example, these relative diameters may be appropriate for implantation at bifurcation(s) between the descending aorta and the renal arteries.

Reference is made to both FIGS. 1 and 2. For some applications, a perpendicular cross-sectional area of a portion of first stent body 32 increases from peripheral juncture 104 in a direction toward a distal end of first stent body 32 (the increasing portion typically does not extend entirely to the distal end). This sloped portion provides a sealing countersurface 116 covered by first fluid flow guide 102 near juncture 104. Typically, at least a portion of sealing countersurface 116 contacts an external surface of second stent-graft 22 surrounding interface aperture 110, when the part of interface portion 103 is positioned within interface aperture 110, as shown in FIG. 2. For some applications, the perpendicular cross-sectional area of the portion of first stent body 32 at an axial distance of from peripheral juncture 104 is at least 30%, such as at least 50%, greater than a perpendicular cross-sectional area of peripheral juncture 104, which axial distance equals 0.3 times an average diameter of first stent body 32, if first and second stent-grafts 20 and 22 were to assume their respective radially-expanded states with interface portion 103 not positioned within interface aperture 110, and no force were exerted on the interface portion by either the first or the second stent-graft. In other words, the interface aperture and the interface portion are characterized by these relative cross-sectional areas when the interface portion is fully radially-expanded, and not constrained by the interface aperture. During actual deployment of prosthesis 10 with the interface portion positioned within the interface aperture, as shown in FIG. 2, the interface aperture sometimes prevents this full radial expansion of the interface portion. In the sealing state, the engagement support members are typically near sealing countersurface 116.

For some applications, after increasing toward the distal end of first stent body 32, the perpendicular cross-sectional area decreases in the direction toward a distal end of first stent body 32 (configuration not shown).

Reference is again made to FIG. 1. For some applications, a perpendicular cross-sectional area of interface aperture 110 is between 65% and 100%, such as between 65% and 85%, e.g., between 65% and 80%, of a greatest outer perpendicular cross-sectional area of interface portion 103, if first and second stent-grafts 20 and 22 were to assume their respective radially-expanded states with interface portion 103 not positioned within interface aperture 110, and no force were exerted on the interface portion by either the first or the second stent-graft. In other words, the interface aperture and the interface portion are characterized by these relative cross-sectional areas when the interface portion is fully radially-expanded, and not constrained by the interface aperture. During actual deployment of prosthesis 10 with the interface portion positioned within the interface aperture, as shown in FIG. 2, the interface aperture sometimes prevents this full radial expansion of the interface portion. The radially-outward force applied by the interface portion against the interface aperture helps provide a tight seal between the first and second stent-grafts.

Reference is made to FIG. 3, which is a schematic illustration of the deployment of two of engagement support members 105, in accordance with an application of the present invention. Four stages of deployment of the engagement support members 105 are indicated with circled numerals 1 through 4. In practice, the engagement support members typically move continuously from (a) an initial state, indicated by numeral 1, in which the engagement support members extend proximally, to (b) a sealing state, indicated by numeral 3, in which the engagement support members extend distally toward a distal end of first stent body 32. For clarity of illustration, only two intermediate positions are shown, indicated by numerals 2 and 3.

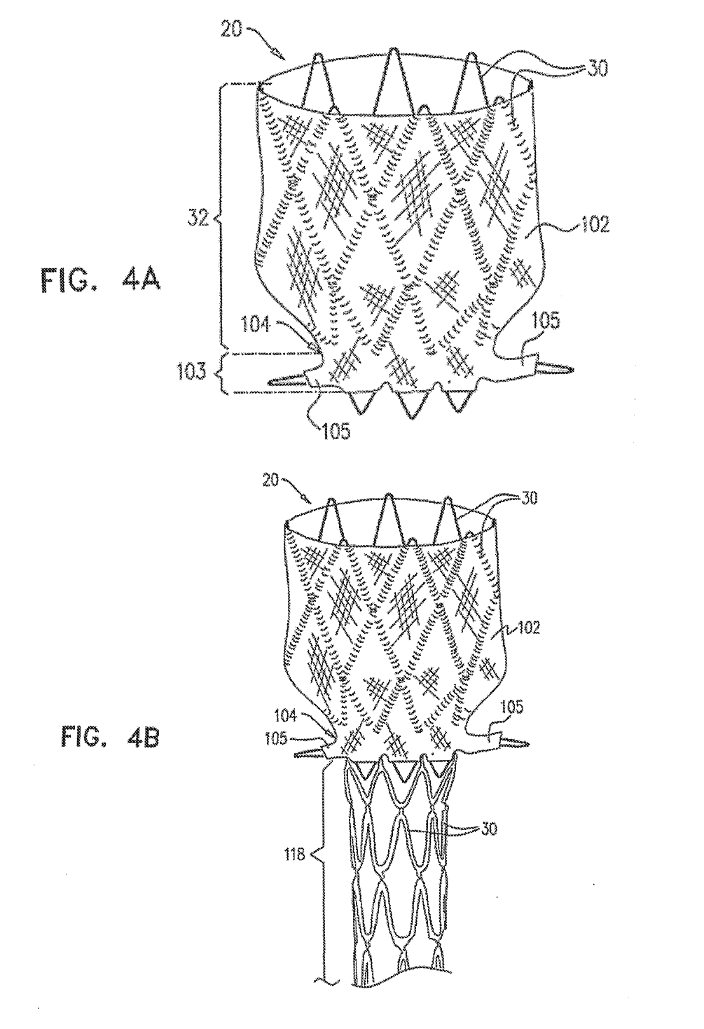

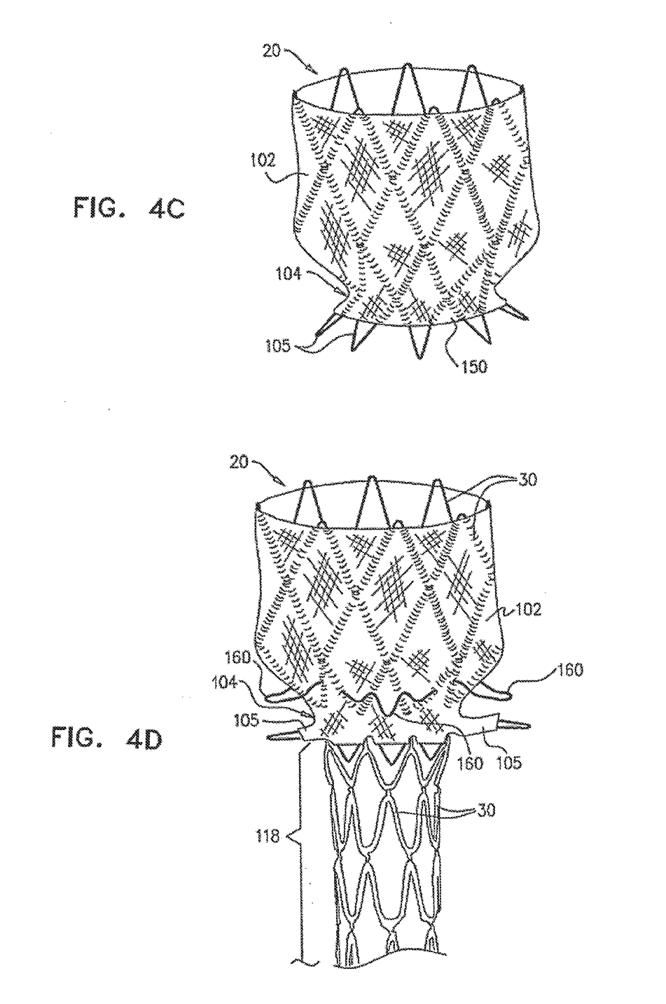

Reference is made to FIGS. 4A-D, which are schematic illustration of additional configurations of first stent-graft 20, in accordance with respective applications of the present invention. In these configurations, first fluid flow guide 102 at least partially covers engagement support members 105. The covered portions help seal first stent-graft 20 to second stent-graft 22, as described hereinabove with reference to FIG. 2, in order to create a continuous, substantially fluid-impervious fluid flow path through the first and second stent-grafts, such as described hereinbelow with reference to FIG. 7.

For some applications, when the first stent-graft assumes its radially-expanded state, engagement support members 105 are shaped so as to define respective radially-inward portions and radially-outward portions, and the first fluid flow guide covers the radially-inward portions but not the radially-outward portions, as shown in FIGS. 4A-D. Alternatively, the first fluid flow guide covers the entirety of the engagement support members (configuration not shown).

For some applications, the covered portion of interface portion 103 extends proximally beyond peripheral juncture 104 by no more than 0.3 times an average diameter of first stent body 32. Alternatively, the covered portion of interface portion 103 extends proximally beyond peripheral juncture 104 by between 0.1 and 0.5 times the average diameter of first stent body 32, as described hereinabove with reference to FIG. 1. In this latter case, the sealing techniques of the configurations shown in FIGS. 4A-D (in which the engagement support members are at least partially covered by the first fluid flow guide) are combined with those described hereinabove with reference to FIG. 1 (in which interface portion 103 may include a substantial covered portion).

Optionally, as shown in FIG. 4B, first stent-graft 20 comprises non-covered portion 118, as described hereinabove with reference to FIG. 1.

For some applications, as shown in FIG. 4C, first stent-graft 20 extends between at least two of the engagement support members that are circumferentially adjacent each other, when the first stent-graft assumes its radially-expanded state. These extension portions 150 (which are similar to webbing) help seal first stent-graft 20 to second stent-graft 22. Optionally, the techniques described with reference to FIG. 4C are combined with the techniques described with reference to FIG. 4B.

Reference is made to FIG. 4C, which is a schematic illustration of another configuration of first stent-graft 20, in accordance with an application of the present invention. For some applications, first stent-graft 20 may comprise an additional set of a plurality of distal engagement support members 160, which are disposed more distally on first stent-graft 20 than are engagement support members 105. Distal engagement support members 160 typically extend from first stent-graft 20 near juncture 104 (typically either slightly distal to the juncture, as shown, or slightly proximal to the juncture). When stent-graft 100 assumes its radially-expanded state, these additional engagement support members extend generally radially outward and/or proximally, against and/or opposite engagement support members 105, so as to sandwich therebetween at least a portion of second fluid flow guide 109 surrounding interface aperture 110 (including periphery 111 of interface aperture 110). Optionally, first stent body 32 is not shaped so as to define sealing countersurface 116. Although the configurations described in this paragraph are shown in combination with the configuration shown in FIG. 4B, these configurations may also be practiced in combination with the configurations shown in FIGS. 1, 4A, and/or 4C.

Alternatively or additionally, sealing countersurface 116, described hereinabove with reference to FIG. 1, is provided by an additional covered element that extends radially outward from first stent-graft 20 near juncture 104 (typically either slightly distal to the juncture, or slightly proximal to the juncture). For example, the additional covered element may be disk-shaped, and comprise a portion of structural stent elements 30, covered by a portion of first fluid flow guide 102 (configuration not shown). The configuration described in this paragraph may be practiced in combination with the configurations shown in FIGS. 1, 4A, 4B, 4C, and/or 4D.

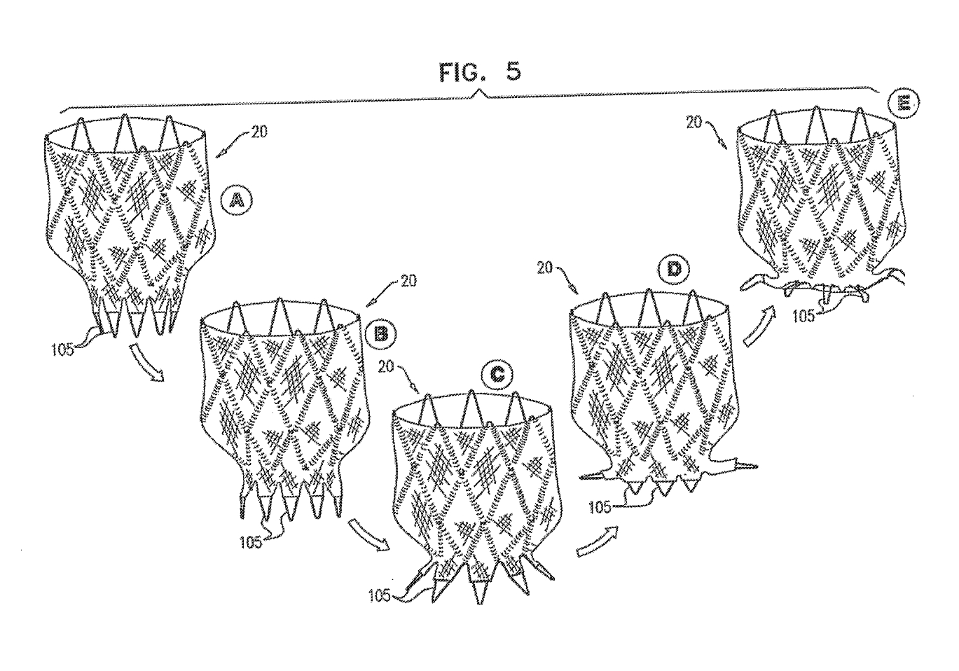

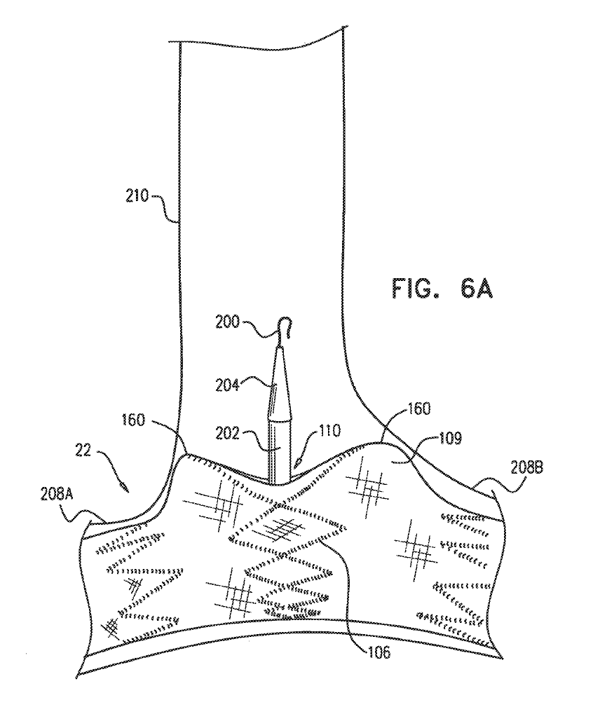

Reference is made to FIG. 5, which is a schematic illustration of the deployment of engagement support members 105 of the configuration of first stent-graft 20 shown in FIGS. 4A-D, in accordance with an application of the present invention. Five stages of deployment of the engagement support members 105 are indicated with circled letter A through E. In practice, the engagement support members typically move continuously from (a) an initial state, indicated by letter A, in which the engagement support members extend proximally, to (b) a sealing state, indicated by letter E, in which the engagement support members extend distally toward a distal end of first stent body 32. For clarity of illustration, only three intermediate positions are shown, indicated by letters B through D. For some applications, engagement support members 105 are inclined only slightly distally. Optionally, a portion (such a most radially-outward portion) of at least some of the engagement support members is distally convex (i.e., convex when viewed from a distal direction), such as to provide a large surface area pressing against second fluid flow guide 109 surrounding aperture 110. This inclination and shape generally reduce the wear and tear that the engagement support members may cause on second fluid flow guide 109. Optionally, the engagement support members additionally extend somewhat radially outward in the initial state, one or more of the intermediary states, and/or sealing state. Alternatively, for some applications, when in the sealing state, the engagement support members do not extend distally, but instead only extend more distally than when in the initial state, and/or extend more radially-outward than when in the initial state (which may be when the first stent-graft is in its radially-compressed state), without necessarily extending more distally than when in the initial state (which may be when the first stent-graft is in its radially-compressed state).

Reference is made to FIGS. 6A-D, which are schematic illustrations of an exemplary method of deploying endovascular prosthesis 10, using an endovascular stent-graft delivery tool, in accordance with an application of the present invention. The method may be used for deploying either the configuration of prosthesis 10 described hereinabove with reference to FIG. 1-3 or 4A-5. The method is shown, by way of example, for deploying second stent-graft 22 in right and left iliac arteries 208A and 208B, and first stent-graft 20 in a descending aorta 210. The method may also be used to deploy prosthesis 10 in other blood vessels, mutatis mutandis, such as described hereinbelow.

The method typically begins with the deployment of second stent-graft 22 in right and left iliac arteries 208A and 208B. Techniques for such deployment are well known in the art, and are thus not shown. Typically, the second stent-graft is transvascularly (typically percutaneously) introduced into one of the iliac arteries, while positioned in its radially-compressed state in a delivery catheter. The second stent-graft is advanced to the other iliac artery, and deployed in both iliac arteries, such that interface aperture 110 is at the aorto-iliac bifurcation.

As shown in FIG. 6A, the delivery tool used for delivering first stent-graft 20 typically comprises a delivery catheter 202, a distal tip 204, and a guidewire 200. In order to implant the first stent-graft, the first stent-graft is transvascularly (typically percutaneously) introduced into the aorta via one of iliac arteries 208, while the stent-graft is in its radially-compressed state positioned in delivery catheter 202. (When the first stent-graft is initially positioned in delivery catheter 202 in its radially-compressed state, the delivery catheter holds engagement support members 105 in their initial state.) Delivery catheter 202 and distal tip 204 are advanced over guidewire 200, and through one of the sides of second stent-graft 22 and interface aperture 110, until the distal tip is positioned in descending aorta 210.

As shown FIGS. 6B and 6C, delivery catheter 202 is withdrawn proximally, allowing first stent-graft 20 to assume its radially-expanded state.

As shown in FIG. 6D, if necessary, first stent-graft 20 is manipulated until part of interface portion 103 is positioned within interface aperture 110, and both the first and second stent-graphs are in their fully-deployed states. As shown in FIG. 2 (but not visible in FIG. 6D), engagement support members 105 have assumed the sealing state, such that the engagement support members sealingly couple first stent-graft 20 to second stent-graft 22, thereby preventing fluid from leaking between the two stent-grafts at interface aperture 110.

For some applications, to aid in the deployment procedure, prosthesis 10 comprises one or more radiopaque markers, disposed on respective ones of the engagement support members (e.g., on the radially-outward portions of the engagement support members, which are described hereinabove with reference to FIGS. 4A-D), and/or on the first structural member.

Reference is again made to FIG. 6A. For some applications, second fluid flow guide 109 is shaped so as to define a radially-outward bulge 160 at least partially surrounding interface aperture 110, when second stent-graft 22 assumes its radially-expanded state. When interface portion 103 is positioned within interface aperture 110, the bulge extends distally toward first stent-graft 20. For applications in which first stent-graft 20 provides countersurface 116, as described hereinabove with reference to FIGS. 1 and 2, the bulge typically contacts the sealing countersurface when the first stent-graft is sealingly coupled to the second stent-graft, as shown in FIG. 6D. For applications in which the second stent-graft is positioned in the iliac arteries, the bulge extends toward the aorto-iliac junction.

Reference is made to FIGS. 6E-F, which schematically illustrate a method for axially elongating prosthesis 10, in accordance with an application of the present invention. In this application, prosthesis 10 comprises a third stent-graft 220, which may be generally similar to first stent body 32 of first stent-graft 20. After first stent-graft 20 has been sealingly coupled to second stent-graft 22, as described hereinabove with reference to FIG. 6D, third stent-graft 220 is introduced into descending aorta 210 while in a radially-compressed state in a delivery catheter (optionally, delivery catheter 202), typically via second stent-graft 22 and first stent-graft 20, as shown in FIG. 6E. A proximal portion of third stent-graft 220 is positioned near a distal end of first stent-graft 20.

As shown in FIG. 6F, when third stent-graft 220 is fully released from the delivery catheter, the third stent-graft assumes a radially-expanded state. The proximal end of the third stent-graft expands radially outward within a distal end of the first stent-graft, thereby sealingly coupling the third stent-graft to the first stent-graft, and creating a continuous fluid flow path through the third and first stent-grafts, such as described hereinbelow with reference to FIG. 7. Additional stent-grafts similar to third stent-graft 220 may be provided and daisy-chained together to further lengthen the fluid flow path.

Another endovascular prosthesis, such as a stent-graft, may be similarly coupled to the distal end of prosthesis 10, such as to first stent-graft 20, third stent-graft 220, or any additional stent-grafts distally connected to prosthesis 10. For some applications, this other endovascular prosthesis is introduced via passage through second stent-graft 22 and first stent-graft 20. For other applications, this other endovascular prosthesis is first introduced, and prosthesis 10 is subsequently introduced and coupled to the other endovascular prosthesis. Alternatively or additionally, first stent-graft 20 may comprise additional elements distal to the distal end thereof shown in the figures, such as branches, anchoring elements, stent elements, and/or fluid flow guides; for example, first-stent graft may implement some of the features described in one or more of the patent applications incorporated hereinbelow by reference.

Reference is made to FIG. 7, which shows an exemplary fluid flow path through endovascular prosthesis 10, in accordance with an application of the present invention. Prosthesis 10 provides this exemplary fluid flow path when the prosthesis is implanted in the descending aorta and iliac arteries, as described hereinabove with reference to FIGS. 6A-D and/or 6E-F. The prosthesis provides other fluid flow paths when implanted at other anatomical locations, such as described hereinbelow. Arrows 230 schematically indicate blood flow through first stent-graft 20 (and, optionally, third stent-graft 220, if provided, as described hereinabove with reference to FIGS. 6E-F). After passing through interface aperture 110 and into second stent-graft 22, the fluid flow path bifurcates, as schematically indicated by a bifurcated arrow 232, such that a portion of the blood flows into right iliac artery 208A, as schematically indicated by an arrow 234A, and the remainder of the blood flows into left iliac artery 208B, as schematically indicated by an arrow 234B.

First fluid flow guide 102 and second fluid flow guide 109 thus together define a continuous fluid flow path that begins at a distal end of first fluid flow guide 102, passes through interface aperture 110, bifurcates proximally to interface aperture 110, and passes through both ends 112 of second fluid flow guide 22. The fluid flow path is continuous because first fluid flow guide 102 is sealingly coupled to second fluid flow guide 109, as described hereinabove. For some applications, endovascular prosthesis 10 is configured such that the fluid flow path provides substantially equal fluid flow through both ends 112 of second fluid flow guide 22.

As mentioned above, for some applications endovascular prosthesis 10 is implanted in the descending aorta and iliac arteries at the aorto-iliac bifurcation. The prosthesis may be also implanted at other bifurcations in the body, such as at other vascular bifurcations. Such other vascular bifurcations include, but are not limited to:
the bifurcation between the descending aorta and one of the renal arteries. Second stent-graft 22 is typically positioned in the descending aorta spanning the bifurcation, and first stent-graft 20 is positioned in the renal artery. Optionally, second stent-graft 22 is shaped so as to define two interface apertures 110, and two first stent-grafts 20 are provided, which are positioned in respective renal arteries. For this application, the fluid flow path begins at one end of the second stent-graft, and bifurcates at the interface aperture(s) into the first stent-graft(s) and the remaining length of the second stent-graft.
the bifurcation between one of the carotid arteries and the internal and/or external carotid artery. Second stent-graft 22 may be positioned in the carotid artery, and first stent-graft 20 may be positioned in the internal or external carotid artery. Optionally, an additional first stent-graft may be positioned in the other of the internal or external carotid artery, in which case the second stent-graft is shaped so as to define two interface apertures. Alternatively, the second stent-graft may be positioned in the external carotid artery and internal carotid artery, spanning the common carotid artery, and the first stent-graft is positioned in the common carotid artery. Further alternatively, the second stent-graft may be positioned in the common carotid artery and either the external or internal carotid artery, and the first stent is positioned in the other of the external or internal carotid artery.
the bifurcations between the aortic arch and the brachiocephalic trunk, common carotid artery, and subclavian artery. Second stent-graft 22 is positioned in the aortic arch, spanning one or more of these bifurcations, and one, two, or three first stent-grafts 20 are positioned in one, two, or three of the brachiocephalic trunk, common carotid artery, and subclavian artery, respectively. Second stent-graft 22 is shaped so as to define one, two, or three interface apertures 110, as appropriate.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
PCT Application PCT/IL2008/000287, filed Mar. 5, 2008, which published as PCT Publication WO 2008/107885 to Shalev et al.
U.S. application Ser. No. 12/529,936, which published as US Patent Application Publication 2010/0063575 to Shalev et al.
U.S. Provisional Application 60/892,885, filed Mar. 5, 2007
U.S. Provisional Application 60/991,726, filed Dec. 2, 2007
U.S. Provisional Application 61/219,758, filed Jun. 23, 2009
U.S. Provisional Application 61/221,074, filed Jun. 28, 2009
U.S. Provisional Application 61/224,089, filed Jul. 9, 2009
U.S. Provisional Application 61/225,228, filed Jul. 14, 2009
PCT Application PCT/IB2010/052861, filed Jun. 23, 2010, entitled, “Vascular prostheses for treating aneurysms”
PCT Application PCT/IL2010/000549, filed Jul. 8, 2010, entitled, “Apparatus for closure of a lumen and methods of using the same”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising an endovascular prosthesis, which comprises:
first and second endovascular stent-grafts, which are configured to transition from respective radially-compressed states to respective radially-expanded states, in which respective radially-expanded states an average diameter of the first stent-graft is greater than an average diameter of the second stent-graft, and which first and second stent-grafts are suitable for implantation at an aorto-iliac bifurcation of a human subject and comprise:
first and second structural members, respectively, which comprise first structural stent elements and second structural stent elements, respectively, at least respective portions of which define first and second stent bodies, which are generally tubular when the first and second stent-grafts assume the respective radially-expanded states; and
first and second fluid flow guides, respectively, which are coupled to the first and second stent bodies, respectively, so as to cover at least respective portions of the first and second stent bodies,
wherein the first structural member has proximal and distal ends, and is shaped so as to define an interface portion having a distal interface end that meets a proximal end of the first stent body at a peripheral juncture,
wherein, when the second stent-graft assumes its radially-expanded state, the second stent-graft is shaped so as to define an interface aperture at a location other than at ends of the second stent-graft,
wherein the interface portion and the interface aperture are configured such that part of the interface portion is positionable within the interface aperture, and
wherein the interface portion comprises a plurality of engagement support members disposed around a periphery of the interface portion, which engagement support members are configured to transition from an initial state to a sealing state, thereby sealingly coupling the first stent-graft to the second stent-graft when the part of the interface portion is positioned within the interface aperture.

2-4. (canceled)

5. The apparatus according to claim 1, wherein a portion of at least some of the engagement support members is distally convex.

6. The apparatus according to claim 7, wherein the engagement support members and the second stent-graft are configured such that the engagement support members internally press against a surface of the bulge of the second stent-graft surrounding the interface aperture, when the part of the interface portion is positioned within the interface aperture, the first and second stent-grafts assume their respective radially-expanded states, and the engagement support members assume the sealing state.

7. The apparatus according to claim 1, wherein the second fluid flow guide is shaped so as to define a radially-outward bulge at least partially surrounding the interface aperture, when the second stent-graft assumes its radially-expanded state.

8-21. (canceled)

22. The apparatus according to claim 1, wherein the engagement support members are configured to extend proximally when in the initial state, and to extend distally toward a distal end of the first stent body when in the sealing state.

23. The apparatus according to claim 1, wherein a perpendicular cross-sectional area of a portion of the first stent body defined by the first structural stent elements and covered by the first fluid flow guide increases from the peripheral juncture in a direction toward a distal end of the first stent body, such that the portion of the first stent body serves as a sealing countersurface, which contacts an external surface of the second stent-graft surrounding the interface aperture when the part of the interface portion is positioned within the interface aperture, the first and second stent-grafts assume their respective radially-expanded states, and the engagement support members assume the sealing state.

24. The apparatus according to claim 23, wherein the engagement support members and the second stent-graft are configured such that the engagement support members and the sealing countersurface sandwich a surface of the second stent-graft surrounding the interface aperture, when the part of the interface portion is positioned within the interface aperture, the first and second stent-grafts assume their respective radially-expanded states, and the engagement support members assume the sealing state.

25-27. (canceled)

28. The apparatus according to claim 1, wherein the first stent-graft comprises a non-covered portion that extends proximally beyond the interface portion, which non-covered portion comprises a portion of the first structural member.

29-30. (canceled)

31. The apparatus according to claim 1, wherein the first fluid flow guide at least partially covers the engagement support members, and wherein, when the first stent-graft assumes its radially-expanded state, the engagement support members are shaped so as to define respective radially-inward portions and radially-outward portions, and wherein the first fluid flow guide covers the radially-inward portions but not the radially outward portions.

32. (canceled)

33. The apparatus according to claim 1,
wherein the engagement support members are proximal engagement support members, wherein the first stent-graft further comprises a plurality of distal engagement support members, which are disposed more distally on the first stent-graft than are the proximal engagement support members, and
wherein the distal and proximal engagement support members are configured to sandwich a surface of the second stent-graft surrounding the interface aperture, when the part of the interface portion is positioned within the interface aperture, the first and second stent-grafts assume their respective radially-expanded states, and the proximal engagement support members assume the sealing state.

34-38. (canceled)

39. The apparatus according to claim 1, wherein the first fluid flow guide and the second fluid flow guide together define a continuous fluid flow path that begins at a distal end of the first fluid flow guide, passes through the interface aperture, bifurcates proximally to the interface aperture, and passes through both ends of the second fluid flow guide.

40. (canceled)

41. Apparatus comprising an endovascular stent-graft, which is configured to transition from a radially-compressed state to a radially-expanded state, and which comprises:
a structural member, which comprises structural stent elements, at least a portion of which defines a stent body, which is generally tubular when the stent-graft assumes the radially-expanded state; and
a fluid flow guide, which is coupled to the stent body, so as to cover at least a portion of the stent body,
wherein the structural member has proximal and distal ends, and is shaped so as to define an interface portion having a distal interface end that meets a proximal end of the stent body at a peripheral juncture,
wherein the interface portion comprises a plurality of engagement support members disposed around a periphery of the interface portion, which engagement support members are configured to transition from an initial state to a sealing state.

42. (canceled)

43. The apparatus according to claim 41, wherein the fluid flow guide at least partially covers the engagement support members, and wherein, when the stent-graft assumes the radially-expanded state, the engagement support members are shaped so as to define respective radially-inward portions and radially-outward portions, and wherein the fluid flow guide covers the radially-inward portions but not the radially outward portions.

44. (canceled)

45. The apparatus according to claim 41, wherein the engagement support members are configured to extend proximally when in the initial state, and to extend distally toward a distal end of the first stent body when in the sealing state.

46-50. (canceled)

51. The apparatus according to claim 41, wherein a perpendicular cross-sectional area of a portion of the stent body defined by the structural stent elements and covered by the fluid flow guide increases from the peripheral juncture in a direction toward a distal end of the stent body, such that the portion of the stent body serves as a sealing countersurface, when the stent-graft assumes the radially-expanded state.

52. The apparatus according to claim 41, wherein the engagement support members are proximal engagement support members, and wherein the stent-graft further comprises a plurality of distal engagement support members, which are disposed more distally on the stent-graft than are the proximal engagement support members.

53. A method comprising:
providing first and second endovascular stent-grafts, which are configured to transition from respective radially-compressed states to respective radially-expanded states, in which respective radially-expanded states an average diameter of the first stent-graft is greater than an average diameter of the second stent-graft, and which first and second stent-grafts include (a) first and second structural members, respectively, which comprise first structural stent elements and second structural stent elements, respectively, at least respective portions of which define first and second stent bodies, respectively, which are generally tubular when the first and second stent-grafts assume the respective radially-expanded states, and (b) first and second fluid flow guides, respectively, which are coupled to the first and second stent bodies, respectively, so as to cover at least respective portions of the first and second stent bodies, wherein the first structural member has proximal and distal ends;
transvascularly introducing the second stent-graft, while in its radially-compressed state, into one or two first blood vessels of a human subject, such that the second stent-graft spans a bifurcation between (a) the one or two first blood vessels and (b) a second blood vessel;
transitioning the second stent-graft to its radially-expanded state, such that an interface aperture defined by the second fluid flow guide is positioned at the bifurcation;
transvascularly introducing the first stent-graft, while in its radially-compressed state, into the second blood vessel via the interface aperture;
positioning, within the interface aperture, an interface portion defined by the first structural member of the first stent-graft, which interface portion has a distal interface end that meets a proximal end of the first stent body at a peripheral juncture; and
transitioning the first stent-graft to its radially-expanded state, such that a plurality of engagement support members thereof, which are disposed around a periphery of the interface portion, transition from an initial state to a sealing state, thereby sealingly coupling the first stent-graft to the second stent-graft.

54. The method according to claim 53, wherein transitioning the first stent-graft comprises transitioning the first stent-graft to its radially-expanded state, such that the plurality of engagement support members transition from (a) the initial state, in which the engagement support members extend proximally, to (b) the sealing state, in which the engagement support members extend distally toward a distal end of the first stent body.

55-56. (canceled)

57. The method according to claim 53, wherein the one or two first blood vessels are right and left iliac arteries, wherein the second blood vessel is a descending aorta, and wherein transvascularly introducing the second stent-graft comprises transvascularly introducing the second stent-graft into the right and left iliac arteries, such that the second stent-graft spans the aorto-iliac bifurcation.

58. (canceled)

59. The method according to claim 61, wherein transitioning the first stent-graft comprises transitioning the first stent-graft to its radially-expanded state such that the engagement support members internally press against a surface of the bulge of the second stent-graft surrounding the interface aperture.

60. (canceled)

61. The method according to claim 53, wherein providing the second fluid flow guide comprises providing the second fluid flow guide shaped so as to define a radially-outward bulge at least partially surrounding the interface aperture, when the second stent-graft assumes its radially-expanded state.

62. (canceled)

63. The method according to claim 53, wherein providing the first stent-graft comprises providing the first stent-graft in which a perpendicular cross-sectional area of a portion of the first stent body defined by the first structural stent elements and covered by the first fluid flow guide increases from the peripheral juncture in a direction toward a distal end of the first stent body, such that the portion of the first stent body serves as a sealing countersurface, and wherein positioning the interface portion and transitioning the first stent-graft to its radially-expanded state comprises causing an external surface of the second stent-graft surrounding the interface aperture to contact the sealing countersurface.

64. The method according to claim 63, wherein causing comprises causing the engagement support members and the sealing countersurface to sandwich a surface of the second stent-graft surrounding the interface aperture.

65. The method according to claim 53, wherein the first stent-graft includes a non-covered portion that extends proximally beyond the interface portion, which non-covered portion comprises a portion of the first structural member, and transvascularly introducing the second stent-graft comprises positioning the non-covered portion in exactly one of the one or two first blood vessels.

66. The method according to claim 53, wherein providing the first stent-graft comprises providing the first stent-graft in which the first fluid flow guide at least partially covers the engagement support members, and, when the first stent-graft assumes its radially-expanded state, the engagement support members are shaped so as to define respective radially-inward portions and radially-outward portions, and the first fluid flow guide covers the radially-inward portions but not the radially outward portions.

67. The method according to claim 53,
wherein providing the first and second stent-grafts comprises providing the first and second stent-grafts in which the first fluid flow guide and the second fluid flow guide together define a continuous fluid flow path that begins at a distal end of the first fluid flow guide, passes through the interface aperture, bifurcates proximally to the interface aperture, and passes through both ends of the second fluid flow guide, and
wherein introducing the second and the first stent-grafts comprises introducing (a) the second stent-graft into the one or two first blood vessels and (b) the first stent-graft into the second blood vessel, such that blood flows into the distal end of the first fluid guide, passes through the interface aperture, and passes out of both the ends of the second fluid flow guide.

68. (canceled)

69. The method according to claim 53, wherein providing the first stent-graft comprises providing the first stent-graft in which a portion of at least some of the engagement support members is distally convex.

70. The method according to claim 53, wherein the engagement support members are configured to extend proximally when in the initial state, and wherein transitioning the first stent-graft to its radially-expanded state comprises transitioning the first stent-graft to its radially-expanded state, such that the engagement support members transition from the initial state to the sealing state in which sealing state the engagement support members extend distally toward a distal end of the first stent body.

71. The method according to claim 53, wherein providing the first stent-graft comprises providing the first stent-graft in which the engagement support members include a portion of first structural stent elements.

72. The method according to claim 61, wherein providing the second fluid flow guide comprises providing the second fluid guide shaped such that a peak of the bulge is farther than the interface aperture from a longitudinal axis of the second stent graft, when the second stent-graft assumes its radially-expanded state.

73. The method according to claim 72, wherein the engagement support members are configured to extend proximally when in the initial state, and wherein transitioning the first stent-graft to its radially-expanded state comprises transitioning the first stent-graft to its radially-expanded state, such that the engagement support members transition from the initial state to the sealing state in which sealing state the engagement support members extend distally toward a distal end of the first stent body.

74. The method according to claim 63, wherein providing the second fluid flow guide comprises providing the second fluid flow guide shaped so as to define a radially-outward bulge at least partially surrounding the interface aperture, when the second stent-graft assumes its radially-expanded state, and wherein transitioning the first stent-graft to its radially-expanded state comprises causing an external surface of the bulge to contact the sealing countersurface.

75. The apparatus according to claim 1, wherein the engagement support members comprise a portion of first structural stent elements.

76. The apparatus according to claim 7, wherein the second fluid flow guide is shaped such that a peak of the bulge is farther than the interface aperture from a longitudinal axis of the second stent graft, when the second stent-graft assumes its radially-expanded state.

77. The apparatus according to claim 76, wherein the engagement support members are configured to extend proximally when in the initial state, and to extend distally toward a distal end of the first stent body when in the sealing state.

78. The apparatus according to claim 23, wherein, when the second stent-graft assumes its radially-expanded state, the second fluid flow guide is shaped so as to define a radially-outward bulge at least partially surrounding the interface aperture, an external surface of which bulge contacts the sealing countersurface when the first stent-graft is sealingly coupled to the second stent-graft.
